(19) United States
(12) Patent Application Publication
(10) Pub. No.: US 2024/0123177 A1
(43) Pub. Date: Apr. 18, 2024

(54) DEVICE FOR CALMING OR MIXING GASES, PROCESS AND COMPUTER PROGRAM FOR MANUFACTURING A DEVICE FOR CALMING OR MIXING GASES

(71) Applicant: Drägerwerk AG & Co. KGaA, Lübeck (DE)

(72) Inventors: Carl-Christoffer NEUMANN, Lübeck (DE); Hendrik FISCHER, Lübeck (DE); Eduard WEBER, Lübeck (DE)

(21) Appl. No.: 18/486,226

(22) Filed: Oct. 13, 2023

(30) Foreign Application Priority Data
Oct. 18, 2022 (DE) 10 2022 127 301.6

(51) Int. Cl. A61M 16/10

(52) U.S. Cl. CPC A61M 16/104; B33Y 80/00

(57) ABSTRACT

Devices (100) for calming or mixing gases are provided. The devices (100) enable calming and/or mixing with an integrated mixing geometry, integration of sensors and actuators, and optimized conduction as well as provision of the mixed gases. The devises include one or more gas inlets, a gas outlet and a mixing system, including the integrated mixing geometry that is manufactured integrally and gas-tight in a manufacturing or joining process which is based on a formation of a form-fitting and/or force-fitting as well as gas-tight connection of plastic materials and/or metallic materials based on a printing technology or 3D printing technology.

### CROSS REFERENCE TO RELATED APPLICATIONS

This application claims the benefit of priority under 35 U.S.C. § 119 of German Application 10 2022 127 301.6, filed Oct. 18, 2022, the entire contents of which are incorporated herein by reference.

### TECHNICAL FIELD

The present invention relates to an embodiment of devices for a calming (flow calming or flow settling) or mixing of gases. The device can be manufactured with a manufacturing or joining process, which is based on a formation of a form-fitting and/or force-fitting as well as gas-tight connection of plastic materials and/or metallic materials using a printing technology or 3D printing technology.

The present invention also relates to a process and computer program for controlling a production facility for manufacturing a device for calming or mixing gases.

### BACKGROUND

Devices for mixing, blending gases, are often also called mixing devices or mixers and are used to mix gases in an anesthesia machine.

Devices for calming gases are often also called devices for harmonization and are used to prepare flow situations for subsequent, i.e. downstream, measuring devices for determining gas states (pressure, flow, flow rates) or gas concentrations, for example in anesthesia devices or ventilators.

Typical gases that are mixed or intermixed in an anesthesia machine are, for example, oxygen (O2), medical compressed air and nitrous oxide, which can be provided at a pressure level of approximately 3.5 bar to 6 bar by means of a central compressed gas supply from the infrastructure of a hospital or by means of compressed gas cylinders.

For a mixture of these gases with calming and/or their reliable homogeneous mixing of the individual gases, for example at a pressure level of 3.5-5 bar, there are general conditions of pressure resistance as well as design requirements for the device for mixing gases.

The design requirements include, in particular, the resistance of the materials used to the gases supplied as well as the size of the overall structure of the device with connecting components and/or elements, supply lines, valves and/or sensors, which should be as small as possible in order to be able to integrate the device for calming or mixing gases into the anesthesia device with minimal space requirements.

Connecting components or elements for connecting the device include, for example, connecting elements which are configured and provided for connecting the hose lines to the central compressed gas supply or compressed gas cylinders.

Connecting components or elements for connecting the device may also include, for example, connecting elements which are configured and provided for connection to the breathing system or breathing circuit of the anesthesia device.

### SUMMARY

With regard to the flow guidance and supply of respiratory gases (breathing gases), there is therefore a need, based on the state of the art, to improve the functionality of devices for calming or mixing gases.

Features and details described in the context of the present inventions in connection with devices and embodiments of devices apply, of course, also in connection with the processes described in the context of the present invention as well as computer programs (provided with non-transitory media) for carrying out the process and their embodiments, as well as vice versa in each case, so that with regard to the disclosure concerning the individual aspects of the invention, reference is or can always be made mutually.

It is an object of the present invention to disclose a device for calming or mixing gases or gas mixtures.

Another object of the present invention is to provide a process for controlling a production device for automated production of a device for calming or mixing gases.

Another object of the present invention is to provide a computer program with non-transitory media (a non-transitory computer-readable medium) or computer program product with non-transitory media (a non-transitory computer-readable medium) that implements automation of manufacturing steps on a production facility.

The objects are attained with a device for a calming or mixing of gases or gas mixtures for an anesthesia system according to the invention, as well as a device for a calming of flowing gases or gas mixtures according to the invention, as well as by a process of automated additive manufacturing of such devices as well as a computer program or computer program product comprising program code provided with non-transitory media for performing at least some aspects of the processes.

The object is attained by a device for a calming gases or gas mixtures or mixing gases or gas mixtures for an anesthesia system that comprises: a mixing system comprising an integrated mixing geometry; a plurality of gas inlets, the gas inlets for each supplying a gas or a gas mixture; a gas outlet for providing an output of a mixed gas. The mixing system is fluidically connectable to at least two gas supply sources by means of the gas inlets and the mixing system is fluidically connectable to at least one mixed gas inlet of a breathing system of the anesthesia system by means of the gas outlet. The device comprising the gas inlets, the gas outlet and the mixing system, comprising the integrated mixing geometry, is manufactured integrally (as an integral single piece component) and gas-tight in a manufacturing or joining process which is based on a formation of a form-fitting and/or force-fitting as well as gas-tight connection of plastic materials and/or metallic materials based on a printing technology or 3D printing technology.

The object is attained by a device for a calming of flowing gases or gas mixtures that comprises: a gas inlet for a supply of a gas or a gas mixture; a flow channel comprising a mixing system comprising an integrated mixing geometry; and a gas outlet for providing a mixed gas output. The device comprising the gas inlet, the gas outlet, the flow channel, comprising the mixing system with the mixing geometry, is manufactured integrally (as an integral single piece component) and gas-tight in a manufacturing or joining process which is based on a formation of a form-fitting and/or force-fitting as well as gas-tight connection of plastic materials and/or metallic materials based on a printing technology or 3D printing technology.

The object is attained by a process for a control of a production device for an automated production of the device for calming or mixing gases that includes forming the device so as to comprise at least two of the following functional components: a gas inlet; another gas inlet; a gas outlet; an interface; an element to settle the flow; a laminar flow element; a flow resistance element; an orifice element; an impact element; and a gas volume buffering element as a common component. The common component is formed in a manufacturing or joining process based on a printing technology or 3D printing technology. Materials made of plastic materials, plastic composites, metal materials or metal composites are used in the manufacturing or joining process.

The object is attained by a computer program or computer program product comprising program code provided with a non-transitory computer-readable media, which program code is executable to perform or control the manufacturing or joining process based on a printing technology or 3D printing technology.

Advantageous embodiments of the invention are explained in more detail in the following description with partial reference to the Figures.

Embodiments of a device for calming or mixing gases are shown.

A first inventive aspect is formed by a device for mixing gases. This device according to the first inventive aspect is configured to mix gases or a mixture of gases to form a mixed gas and to provide the mixed gas. The device for mixing gases or gas mixtures for an anesthesia system comprises a mixing system, at least two gas inlets (inputs) for a supply of at least two gases or gas mixtures and at least one gas outlet (output) for a supply of a mixed gas. The mixing system has an integrated mixing geometry. The mixing system is fluidically connectable to at least two gas supply sources by means of the gas inlet. The mixing system is fluidically connectable to at least one mixed gas inlet (gas input) of a breathing system of the anesthesia system by means of the at least one gas outlet (gas output). The device with the gas inlets, the gas outlet and the mixing system with the mixing geometry can be integrally manufactured (as a one piece structure—single component) in a gas-tight manner in a manufacturing or joining process, which is based on a formation of a form-fitting and/or force-fitting as well as gas-tight connection of plastic materials and/or metallic materials using (based on) a printing technology or using (based on) 3D printing technology. The device for mixing gases enables a mixture or blending of at least two gases by means of the mixing geometry to form a gas mixture as a mixed gas and the provision of the mixed gas at the gas outlet.

A further inventive aspect is formed by a device for calming (settling) a flowing gas or gas mixture. A device for calming gases comprises a flow channel with a mixing system, at least one gas inlet for a supply of a gas or gas mixture and a gas outlet for a supply of a mixed gas. The mixing system has an integrated mixing geometry which settles/calms the flowing gas or gas mixture by means of different configurations of elements for flow calming.

According to the invention, the device for calming gases makes it possible to calm flows of a gas mixture in a flow channel by means of the mixing geometry to form a mixed gas and to provide the mixed gas at the gas outlet. In this way, turbulent flows in the flow channel can be homogenized, made uniform or laminarized, for example, and excessive flow velocities present at the edges (peripheral areas) or in the center of the flow channel can be reduced. As a result, a reproducible profile of the flow velocity over the flow cross-section of the flow channel can be achieved. The device with the gas inlet, the gas outlet, the flow channel and the mixing system with the mixing geometry can be manufactured integrally in a gas-tight manner in a manufacturing or joining process which is based on the formation of a form-fitting and/or force-fitting as well as gas-tight connection of plastic materials and/or metallic materials based on a printing technology or based on 3D printing technology. The integral production (integral—one piece—manufacturing) is carried out with a manufacturing or joining process which is based on the formation of a form-fitting and/or force-fitting as well as gas-tight connection of plastic materials and/or metallic materials using a printing technology or using 3D printing technology.

The gas mixture provided as a mixed gas can advantageously be used for performing inhalation anesthesia on a living being or patient, for example, by an anesthesia system or anesthesia machine.

The integral configuration (one piece structure—single component) of the mixing system offers the advantages of configuring the gas mixture in an anesthesia system in such a way that the smallest possible number of pneumatic interfaces is required for embedding the mixing system in the anesthesia system. For example, the requirement for the use of hose connections with associated plug/coupling connections can be reduced, since the integral three-dimensional configuration (one piece structure—single component—three-dimensional configuration) allows the connections of functional elements, which in the anesthesia system serve, for example, flow harmonization or flow calming, such as volume elements for buffering defined gas quantities or elements configured as flow resistors, for example laminar flow elements (LFE) or orifice elements for creating defined pressure situations, can be configured directly and integrally at the gas inlets, in the mixing system or at the gas outlet, so that additional connecting elements, such as pipe or hose connections with plug/coupling elements, are not required. In this way, the number of pneumatic interfaces can be kept as low as possible by means of three-dimensional configuration in 3D printing. This low number of pneumatic interfaces means that the number of leaks at interfaces can also be reduced. In addition, the form factor that must be configured and provided for the gas mixture in the anesthesia system can be minimized in this way, so that reductions in size and construction volume for the entire anesthesia system are also associated with this. In addition, the amount of gases contained in the mixing system during operation can be reduced, which then enables rapid changes in gas concentrations in the gas mixture at the gas outlet compared to non-integral mixing systems. If integrated interfaces, for example pressure sensors, are arranged directly in or on the device via the mixing system, the gas outlet or the gas inlets, otherwise common spur lines from the measuring point to the pressure sensor can be omitted. Again, this reduces the form factor and thus the size to be reserved in the anesthesia system and, moreover, also reduces the number of parts, the assembly time and also the likelihood of leaks and malfunctions based thereon. The assembly time can also be reduced and the assembly itself simplified, which in turn can have a reduction in assembly errors as a positive consequence.

Some embodiments show that individual components or functional assemblies of components of the device for calming or mixing gases can be manufactured in a manufacturing or joining process, which is based on a printing technology or 3D printing technology to form a form-fitting and/or force-fitting as well as gas-tight connection of plastic materials and/or metallic materials. Other materials include plastic composite materials or metal composite materials.

Printing technologies or 3D printing technologies enable the production of parts and/or components in a so-called additive manufacturing process (AM: Additive Manufacturing), i.e. parts and components grow additively layer by layer or layer by layer. Various printing technologies, in particular 3D printing technologies, enable the configuration of shapes that are not possible with machining or milling manufacturing processes or even with conventional injection molding processes.

The particular advantage of the embodiments described, in which printing technologies or 3D printing technologies are used, results from the fact that the components and/or functional assemblies can be configured with a novel and/or also with improved functionality thanks to the new possibilities for shaping.

As examples of other additive manufacturing processes, some additive manufacturing processes are listed below and described in brief.

FDM 3D printing (fused deposition modeling) or fused filament fabrication (FFF), often also referred to as fused layering, refers to a manufacturing process by which a workpiece is built up layer by layer from a fusible plastic or molten metal.

Binder jetting or binder jetting 3D refers to an additive manufacturing process in which powdered starting material is bonded with a binder at selected points to produce workpieces. Subsequent removal of the binder by means of a subsequent sintering process can improve the mechanical properties of the workpieces.

SLS (Selective Laser Sintering), the process of a selective laser sintering (SLS) is an industrial 3D printing process ideal for manufacturing end-use parts. In SLS, a laser selectively sinter polymer powder particles, fusing them and building a part layer by layer.

Stereolithography—a workpiece is built up layer by layer from a light-curing plastic (photopolymers, for example acrylic, epoxy or vinyl ester resins) using stereolithography by means of (raster) dots ((grid) dots) materializing freely in space and cured layer by layer by a laser. In stereolithography processes, large components, since the resin cured by the laser is still relatively soft, and also certain mold elements (e.g. overhangs) must be securely fixed during the building process. For this purpose, support structures are also built during the manufacturing process. After the build process, the components are freed from the support structures, washed with solvents and fully cured in a cabinet under UV light. In microstereolithography for smaller components, no support structures are required, and in many cases post-curing can also be omitted.

MJM (MultiJet Modeling) In MJM processes, parts are produced by spraying a binder onto thin layers of polymer powder particles followed by a sintering process using an IR heat source. MJM produces functional plastic parts with isotropic, mechanical properties that can be used for prototyping or low-volume production end-use applications.

Other additive manufacturing processes are listed, for example, in the German standard “Additive Manufacturing Processes”: VDI 34005, as well as in the US standard “Additive Manufacturing Technologies”: ASTM F42 or in the international standard “Additive Manufacturing”: ISO/TC 261:. Here is an exemplary excerpt without any claim to completeness:
Stereolithography (SL, SLA)
Laser sintering (LS)
Laser beam melting (SLM=Selective Laser Melting, also: Laser Beam Melting=LBM)
Electron Beam Melting (EBM)
Fused Layer Modeling/Manufacturing (FLM)
Fused Filament Fabrication (FFF)
Multi-Jet Modeling (MJM)
Poly-Jet Modeling (PJM)
Binder Jetting
3D printing
Layer Laminated Manufacturing (LLM)
Digital Light Processing (DLP)
Thermal Transfer Sintering (TTS)

In a preferred embodiment, the mixing system may include at least one interface for a connection to sensors, actuators, or a connection to a gas state measurement.

The actuators can be configured, for example, as valves which, as active valves, electrically or pneumatically controlled via a control system, allow gas quantities to enter the mixing system at the gas inlets or gas quantities of the gas mixture to exit the mixing system via the gas outlet. Passive valves may be arranged in the mixing system to prevent backflow of gas quantities. In preferred embodiments, the sensors may be pressure sensors to measure pressure levels at the gas inlets, in the mixing system, or at the gas outlet.

In preferred embodiments, the sensors may be flow rate sensors or flow sensors, such as ultrasonic flow sensors, differential pressure sensors, or anemometric sensors, to measure flow situations, flow rates, flow volumes, or flow velocities at the gas inlets, in the mixing system, or at the gas outlet.

In a further preferred embodiment, an element for flow calming, for example as a flow resistance element or element for volume buffering, is arranged in the mixing system or in or at at least one of the at least one interface.

In implementations of these embodiments, a flow resistance or flow resistance element may be arranged in the mixing system or in or at at least one of the at least one interface. A flow resistance or a flow resistance element can be configured, for example, as an orifice or orifice element for generating a defined pressure drop or a defined pressure difference.

In a further preferred embodiment, a laminar flow element (LFE) may be disposed in the mixing system or in or at at least one of the at least one interface. An LFE represents a special case of a flow resistor. The principle of the LFE is based on Poiseuille's law, according to which a laminar flow in a thin tube or a tube bundle of thin tubes, behaves proportionally to the pressure loss per unit length of the tube.

In a preferred embodiment, the interface can be configured for pressure measurement. The pressure measurement can be realized by a pneumatic and gas-tight coupling of a pressure sensor in the form of a fit, joint connection, plug-in connection, threaded connection, in an advantageous manner with additional sealing elements such as O-rings, to configurations of O-ring plug-in connection, O-ring threaded connection.

In a further preferred embodiment, an element for a volume buffering is arranged in or at at least one of the at least one interface. An element for a volume buffering can be arranged as a space, a cavern, defined, delimited volumes in the mixing system in, at, before or after the gas inlets or the gas outlet.

In a further preferred embodiment, the gas outlet can be configured on the inside with a geometry that forms an inner contour so that there is no step or shoulder in the transition between the gas outlet and the breathing system. The inner contour can be produced in a manufacturing or joining process based on a printing technology or 3D printing technology, or can be produced based on the printing technology or 3D printing technology. A flow condition without a flow stall and/or with an essentially locally laminar flow at the transition of the gas outlet to the breathing system thus reduce possible turbulence and pressure drops and, for example, also possible noise caused by flows.

In a preferred embodiment, the gas inlets or the gas outlet can be configured with a cylindrical or tubular outer contour on the outside. The cylindrical or tubular outer contour is configured to attach or receive a connecting element or a connecting element. A connecting element or connecting element can be configured, for example, as an element which can be connected to the cylindrical or tubular outer contour of the gas outlet by means of a joining connection, a clamping connection, or a cutting connection. One example of this is the so-called cutting ring fittings (cutting ring screw connections). Cutting ring fittings or also cutting ring pipe fittings are assembly elements which are configured and intended to realize gas-tight connections between cylindrical piping elements. Cutting ring fittings are listed, for example, in the International Standard “Metallic tube fittings for fluid power and general use”: ISO 8434-1 and are classified for applications in different pressure ranges. The outer contour can be produced in a manufacturing or joining process based on printing technology or 3D printing technology, or can be produced based on printing technology or 3D printing technology.

In a preferred embodiment, the device may be configured with the gas inlets, the mixing system, the gas outlet and the interfaces as a common component (a single piece integral component). In this case, at least two of the following functional components:
Gas inlets
Gas outlet
Interfaces
Elements to settle (calm) the flow
Laminar flow element
Flow resistance element
Aperture element
Impact element
Elements for volume buffering
   are configured as integral elements (elements provided as a one piece structure) of the mixing system and together form a common component that can be produced in a manufacturing or joining process based on a printing technology or 3D printing technology, and wherein materials made of plastic materials, plastic composites, metal materials or metal composites are used in the manufacturing or joining process based on a printing technology or 3D printing technology.

In a preferred embodiment, the device may be formed with the gas inlets, mixing system, gas outlet, and/or interfaces having an internal contour such that there are no steps or shoulder at a transition to a connector or connecting element.

In a preferred embodiment, the device may form a cylindrical or tubular outer contour on the outside of the gas inlets, mixing system, gas outlet, and/or interfaces.

In a preferred embodiment, the device can form an internal contour on the outside of the gas inlets, mixing system, gas outlet and/or interfaces so that there is no step or shoulder. This enables a flow condition without a stall and/or a substantially locally laminar flow at the transition of the gas outlet to the breathing system to result for a supply of quantities of gas from the gas outlet to the breathing system.

In a further preferred embodiment, the common component can preferably be manufactured in one piece in a manufacturing or joining process based on a printing technology or 3D printing technology, or can be manufactured on the basis of the printing technology or 3D printing technology, wherein materials made of plastic materials, plastic composites, metal materials or metal composites are used in the manufacturing or joining process based on a printing technology or 3D printing technology.

The aspect of the invention relating to a device for calming or mixing gases has been explained above. Hereinafter, another aspect of the invention will be explained in more detail with respect to processes for controlling a production facility for automated production of the device for stilling gases or mixing gases with improved flow control. In addition, an aspect relating to a computer program or computer program product will be explained. The computer program or computer program product enables implementation of the process in an automation of manufacturing steps at a production facility. In particular, printing apparatuses, 3D printing apparatuses as well as drilling, turning or milling apparatuses for a machining or finishing of the device according to the invention prepared by means of the printing apparatuses, 3D printing apparatuses are mentioned here as suitable production devices for the automated production of a device for calming or mixing gases. The printing apparatuses and 3D printing apparatuses enable the production of parts according to the processes already mentioned in the context of this application, such as FDM 3D printing (Fused Deposition Modeling) or Fused Filament Fabrication (FFF), Selective Laser Sintering (SLS), MultiJet Modeling (MJM), Poly-Jet Modeling (PJM), Fused Layer Modeling/Manufacturing (FLM), Selective Laser Melting (SLM), Stereolithography (SL), Laser-Sintering (LS), Electron Beam Melting (EBM). Plastic materials, plastic composites, metal materials or metal composites can be used. The drilling, turning or milling equipment, especially in the form of machine tools with CNC control, (CNC=Computerized Numerical Control) enables automated processing with shaping, surface treatment, drilling, milling, threading of components. The definition of component machining or reworking can be automated by means of a CAM system (Computer-aided manufacturing=CAM). Plastic materials, plastic composites, metal materials or metal composites can be used. A control can include closed-loop control, open-loop control or setting/adjustment of the printing and 3D printing equipment used, as well as drilling, turning or milling equipment.

In a preferred embodiment, a manufacturing or joining process for providing a device for calming or mixing gases is configured and executable as a process for automated additive manufacturing. Such a process enables automated additive manufacturing of a one-piece mixing system with at least two of the following functional components:
Gas inlets
Gas outlet
Interfaces
Elements to settle (calm) the flow
Laminar flow element
Flow resistance element, flow resistance
Aperture element, aperture
Impact element, impact plate
Elements to a volume buffering
   as a common component.

In a preferred embodiment, no support structures are used in additive manufacturing in the manufacturing or joining process based on 3D printing technology for forming the gas inlet, the gas inlets and/or the interfaces. This offers the advantage that a post-processing step can be omitted. Support structures can be omitted, for example, if instead of an essentially circular inner shape, for example of interfaces, gas inlets and/or the gas outlet, an inner contour with a teardrop-shaped or triangular structure is selected.

In a preferred embodiment, support structures are used in the manufacturing or joining process based on 3D printing technology to form the flow calming elements (laminar flow element, flow resistance element, flow resistance, orifice element, orifice plate, baffle element, baffle plate) to form the effect of flow calming in additive manufacturing. In addition to the advantages in additive 3D printing manufacturing, the support structures in the form of a truss or supporting structure, directly form the desired flow obstacles to create a flow calming effect. These support structures can remain in the 3D component as elements for flow calming, and the next manufacturing step with removal of the support structures on these elements can be omitted accordingly.

Thus, in embodiments, the manufacturing or joining process based on 3D printing technology can enable configuration forms to be selected such that for some selected sub-elements of the 3D printed structures, the support structures are unnecessary in the manufacturing process, and for other selected sub-elements, the support structures can be incorporated into the function of the 3D printed structures.

In a preferred embodiment, the process enables automated additive manufacturing in a manufacturing or joining process based on a printing technology or 3D printing technology to form the gas outlet and/or gas inlets with an internal contour so that there is no step or shoulder at a transition to a connecting element or connecting element.

In a preferred embodiment, the process enables automated additive manufacturing in a manufacturing or joining process based on a printing technology or 3D printing technology to form the gas inlets and/or the gas outlet with a cylindrical or tubular outer contour.

In a preferred embodiment, the process enables automated additive manufacturing in a manufacturing or joining process based on a printing technology or 3D printing technology, to form the gas outlet with an inner contour so that there is no step or shoulder. This enables a flow condition without a flow stall and/or with a substantially locally laminar flow at the transition of the gas outlet to the breathing system to result for a supply of quantities of gas from the gas outlet to the breathing system (respiratory system).

In a further embodiment, a computer program (provided as/with a non-transitory computer-readable medium) or computer program product (provided as/with non-transitory computer-readable medium) is formed with program code for performing at least one of the previously described processes to automated additive manufacturing of a device for quietening (settling/calming) or mixing gases when the program code is executable on a computer, a processor, or a programmable hardware component. The computer program or computer program product is formed with program code for executing the process to control a production device, wherein the program code is executable on a computer, a processor, or a programmable hardware component. In this context, the program code—in addition to instructions for controlling the 3D printing apparatus and/or drilling, turning or milling devices—also has data on the shape and configuration of the device for calming or mixing gases with an arrangement having an inner and outer chamber, gas outlet, gas inlet and the second gas supply line. These data may include CAD models, 3D models, 2D models, wireframe models, or vector data on configuration and construction of appropriate computer-aided engineering (CAE) programs. This data may include CAD models, 3D models, 2D models, wireframe models or vector data for computer-aided manufacturing (CAM).

The computer program product and the program code comprise the data required for the creation of the device for calming or mixing gases (CAE, CAM) in order to manufacture it by means of automated additive manufacturing on printing equipment and 3D printing equipment as well as drilling, turning or milling equipment.

On the basis of the following descriptions, the aspects of the invention are explained in more detail, with partial reference to the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a device for gas mixing;

FIG. 2 is a schematic view showing another device for gas mixing;

FIG. 3 is a schematic view showing a device for flow calming;

FIG. 4 is a schematic view showing interfaces and arrangements for flow calming;

FIG. 5 is a schematic view showing interfaces for coupling sensors or actuators;

FIG. 6 is schematic views showing various embodiments of internal structures;

FIG. 7 is a cross sectional view showing a connecting element connected to the device for gas mixing;

FIG. 8 is a cross sectional view and perspective view showing the connecting element according to FIG. 7;

FIG. 9 is schematic views showing configurations of elements for flow calming;

FIG. 10 is perspective views showing configuration aspects of an element for flow calming;

FIG. 11 is a perspective view and a detail view showing configuration aspects of an elements for flow calming; and

FIG. 12 is a perspective view and a detail view showing configuration aspects of an elements for flow calming.

### DESCRIPTION OF PREFERRED EMBODIMENTS

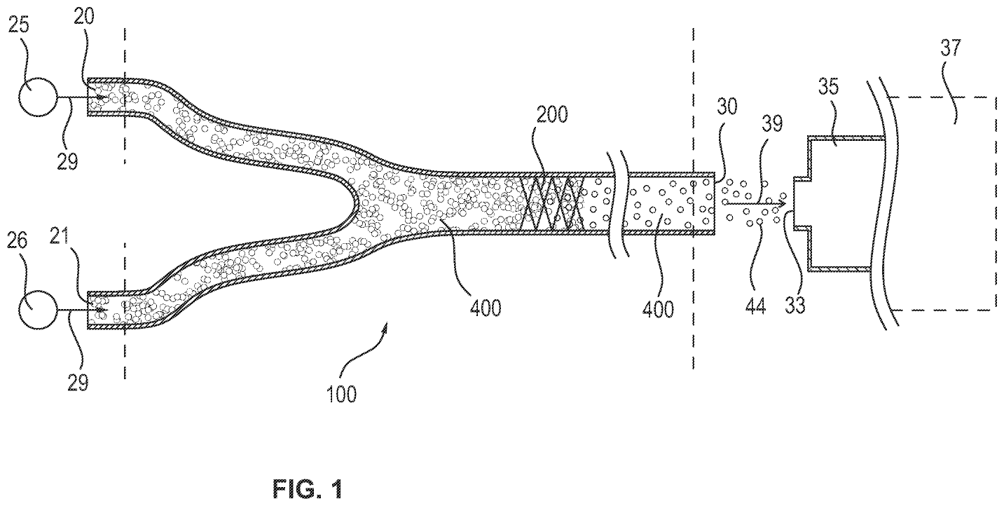

Referring to the drawings, in FIGS. 1 to 10, the directions of flows in device to a gas mixture, in devices or arrangements to a calming of a gas state, are shown by flow arrows. Shown are flow arrows of inflowing gases 29 and flow arrows of outflowing gases 39.

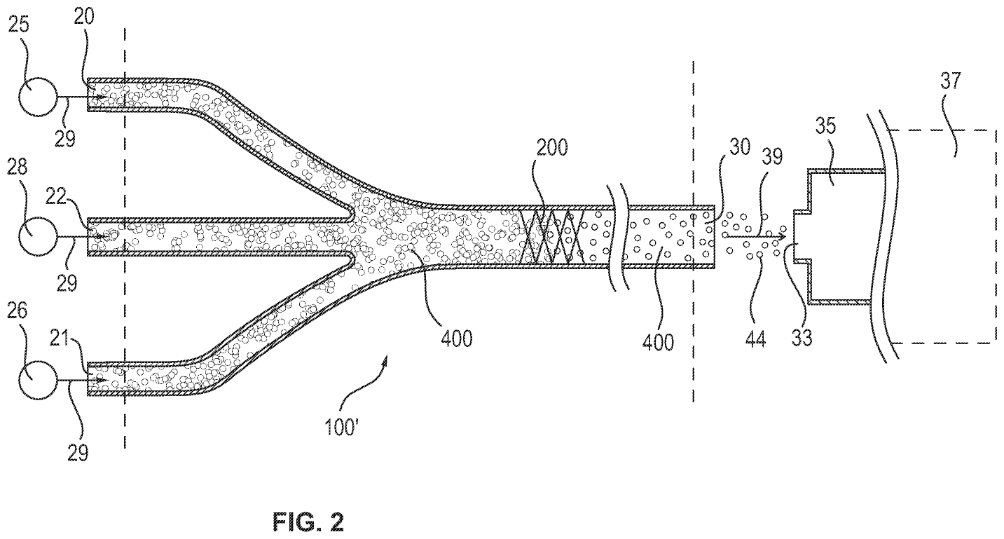

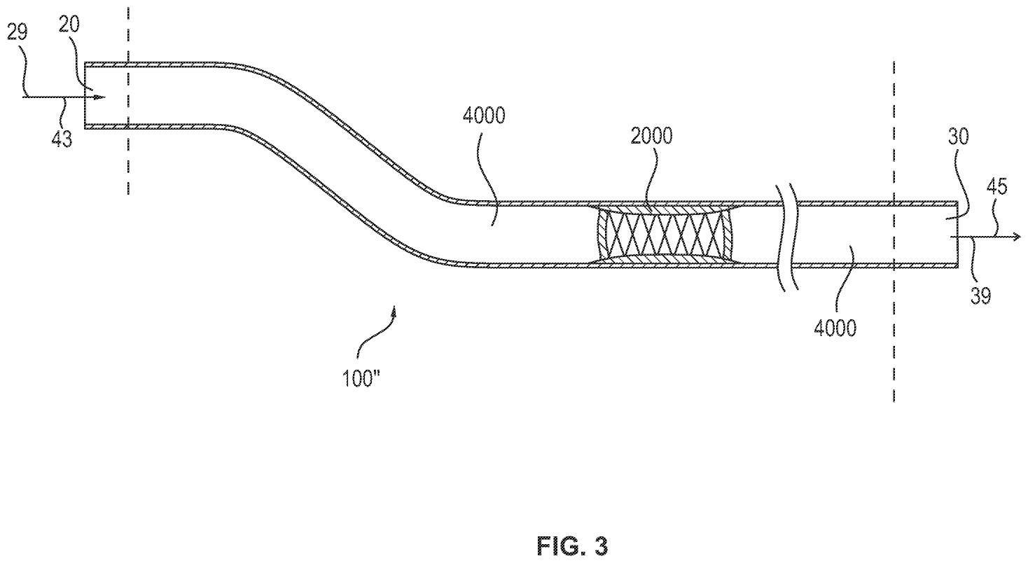

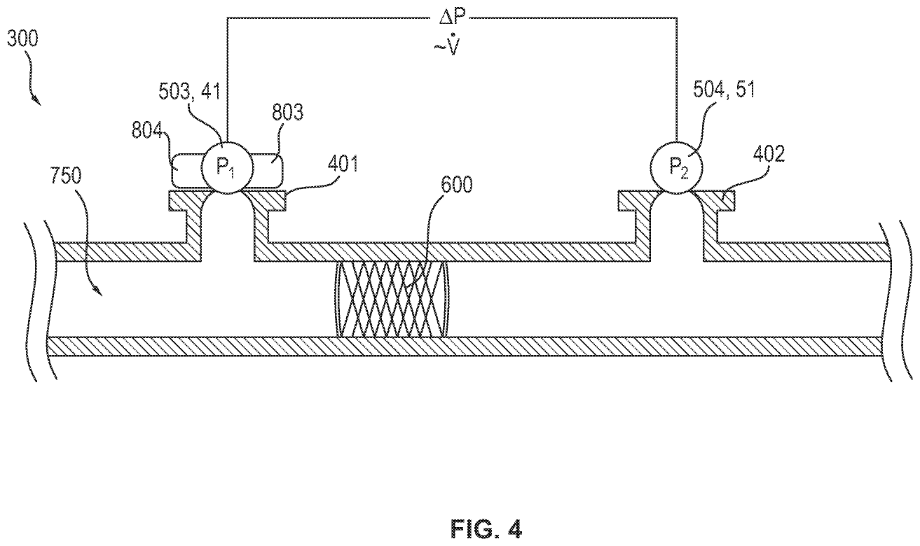

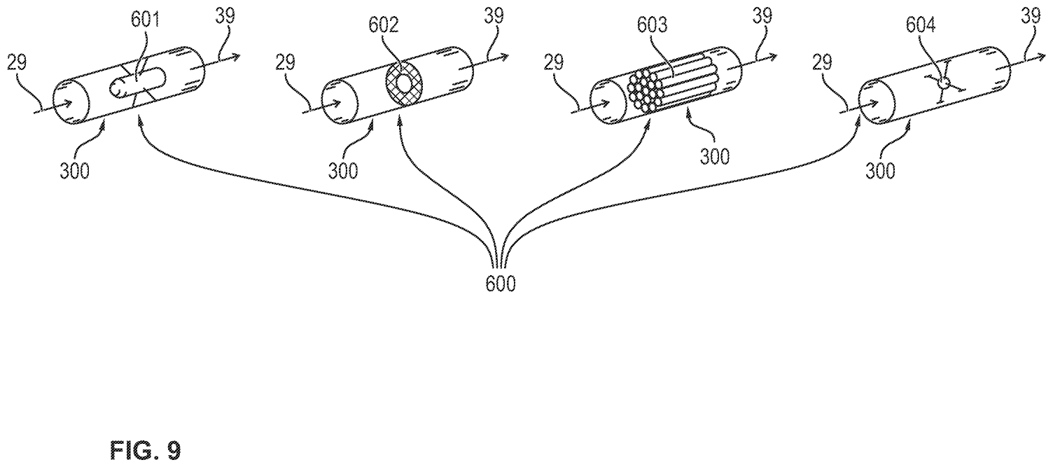

FIG. 1 schematically shows a device 100 for gas mixing with a mixing system 400. Two gas inlets 20, 21 are shown, which are configured and suitable for connecting two gas supply sources 25, 26. Gas supply sources 25, 26 may be, for example, oxygen, either by means of supply through a central gas supply system (CCS) or a compressed gas cylinder, and medical compressed air connected to the gas inlets 20, 21. A mixing geometry 200 with flow calming elements 600 (FIGS. 4, 9, 10, 11, 12) allows mixing of the gases provided at the gas inlets 20, 21 to form a mixed gas 44. The mixed gas 44 can be provided by means of a gas outlet 30 to a breathing system 35 in an anesthesia system 37, anesthesia machine, or ventilator. Various embodiments of flow calming elements 600 (FIG. 4) for use in the mixing geometries 200 (FIGS. 1 and 2), 2000 (FIG. 3) according to FIGS. 1 to 3 are illustrated in FIGS. 9 to 12.

FIG. 2 shows an extension of the device 100 with a mixing system 400 according to FIG. 1 as a further device 100′. Identical elements in FIG. 1 and FIG. 2 are designated with the same reference numerals in FIGS. 1, 2. Three gas inlets 20, 21, 22 are shown, which are configured for connection of two gas supply sources 25, 26, 28. Oxygen, medical compressed air and nitrous oxide can be connected to the gas inlets 20, 21, 22 as gas supply sources 25, 26, 28. A mixing geometry 200 allows mixing of the three gases in the mixing system 400 to form a mixed gas 44. The mixed gas 44 can be provided to a breathing system 35 in an anesthesia system 37 by means of a gas outlet 30.

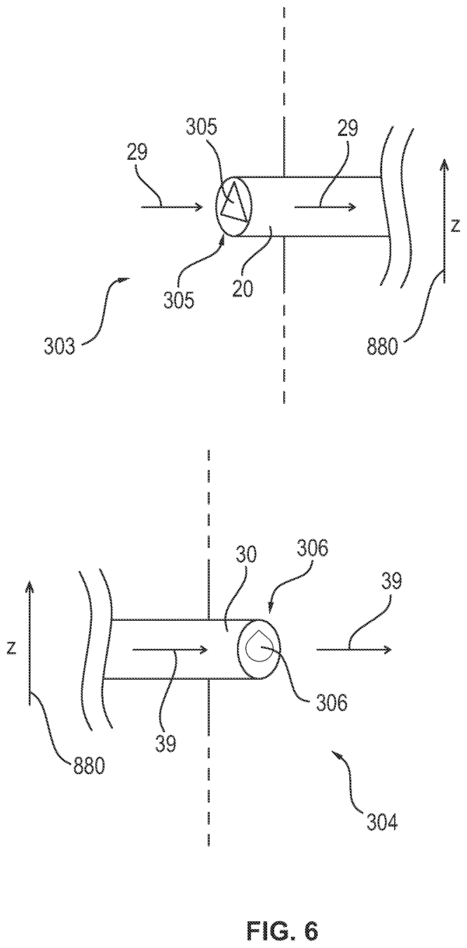

FIG. 3 schematically shows a device 100″ for flow calming or for calming a gas state. Identical elements in FIGS. 1, 2 and FIG. 3 are designated with the same reference numerals in FIGS. 1, 2, 3. A gas inlet 20 and a gas outlet 30 are shown. In a mixing system 4000, a mixing geometry 2000 allows an inlet gas 43 provided at the gas inlet 20 to be settled (calmed), which is then available at the gas outlet 30 as a settled outlet gas 45. The gas inlet 20 and gas outlet 30 are shown in FIGS. 1 through 3 as having a circular shape on the exterior and interior, respectively, with no contours or shaping. Details of an internal structure or possible internal shapes are shown schematically in FIG. 6.

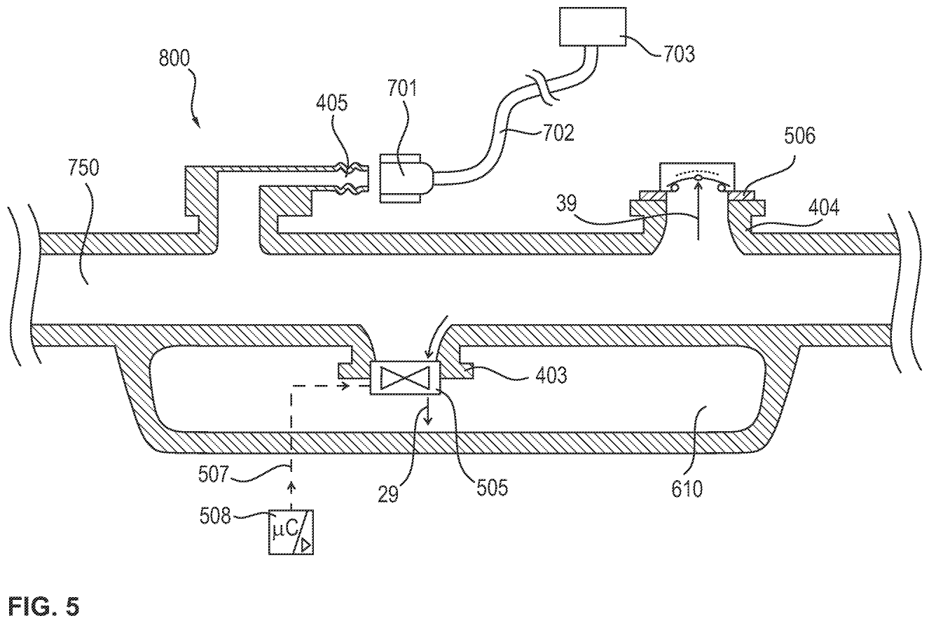

FIGS. 4 and 5 show in schematic representations some examples of arrangements of components for devices 100, 100′, 100″ for a flow calming or calming or mixing of gases or gas mixtures or a mixing system 400, 4000 according to any of FIGS. 1 to 3.

FIG. 4 shows interfaces 401, 402 and shows an exemplary coupling of sensors 501, 502, 503, 504 as well as actuators 505, 506 in an arrangement 300.

Identical elements in FIGS. 1, 2, 3 and FIG. 4 are designated by the same reference numerals in FIGS. 1, 2, 3, 4. Upstream and downstream of a mixing geometry 200 in a flow channel 750, a first and a second interface 401, 402 are arranged. A first pressure sensor (P₁) 503 for detecting a first pressure level P₁, 41 is arranged at the first interface 401, and a second pressure sensor (P₂) 504 for detecting a first pressure level P₂, 51 is arranged at the second interface 402. In this FIG. 4, a temperature sensor (9) 803 and also a humidity sensor (rel. Hum. %) 804 are additionally arranged at the first interface 401 as an example. By means of the two pressure sensors 503, 504 it is possible, for example, to determine a pressure difference (ΔP) across an element 600 for flow calming. Such a pressure difference can be used as a measure of a flow rate through the arrangement 300. The signals from temperature sensor 803 and humidity sensor 804 can be used to convert ambient conditions (humidity, temperature in gas or gas mixture) during the measurement situation to normalized standard conditions, such as STPD (standard temperature, pressure, dry: T=273.15 K, 0° C.), p=101 kPa, water vapor partial pressure p(H₂O)=0 kPa) or BTPS (body temperature, pressure, saturated), ATPS (ambient temperature, pressure, saturated).

FIG. 5 shows interfaces 403, 404, 405 to and with an exemplary coupling of actuators 505, 506 and to the connection 701 of a gas state measurement.

Identical elements in FIGS. 1, 2, 3, 4 and FIG. 5 are designated by the same reference numerals in FIGS. 1, 2, 3, 4, 5. Shown is an arrangement 800 with interfaces 403, 404, 405. Parallel to a flow channel 750, an element 610 is arranged for volume buffering, often also referred to as buffer volume. An actuator 505 in the form of a controllable valve is arranged at an interface 403 in the flow channel 750, which allows gas volumes to be directed from the flow channel 750 into the element 610 for volume buffering. The control of such a valve 505 can be coordinated by a control unit 508 by means of a control line 507. A further actuator 506 is arranged at a further interface 404. This further actuator 506 may, for example, be configured as a passive valve, such as a pressure relief valve or a check valve. A further interface 405 enables a connection 701 by means of a sample gas line 702 to a unit for a gas state measurement unit 703. The state measurement unit 703 may be configured, for example, to determine concentrations of constituents in a gas mixture in the flow channel 750. The interfaces 403, 404, 405 can be integrally formed gas-tight in a manufacturing or joining process based on a formation of a form-fit and/or force-fit as well as gas-tight connection of plastic materials and/or metallic materials on a printing technology or 3D printing technology. The element 610 for a volume buffering can be arranged directly at the flow channel 750 by the integral arrangement 800, so that additional hose lines for coupling the element 610 can be omitted. In this way, an element 610 clearly defined in volume can be coupled to the arrangement 800 in a defined and reproducible manner with flow channel 750.

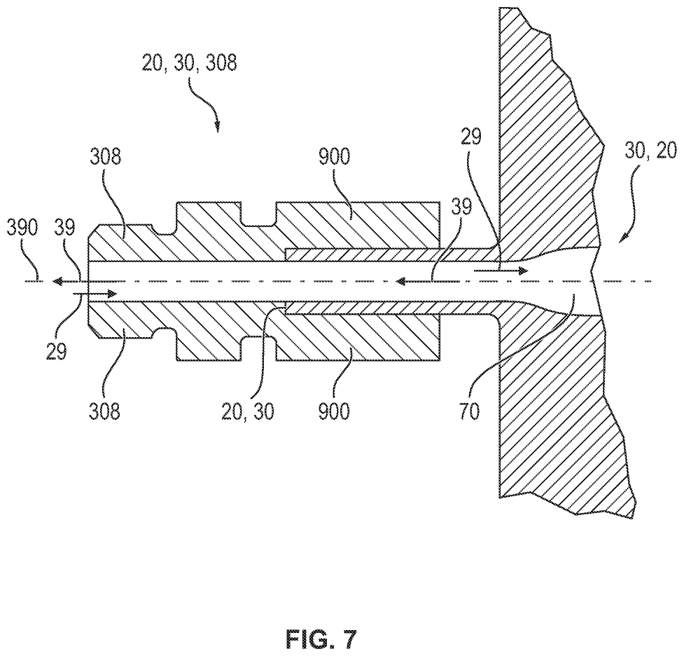

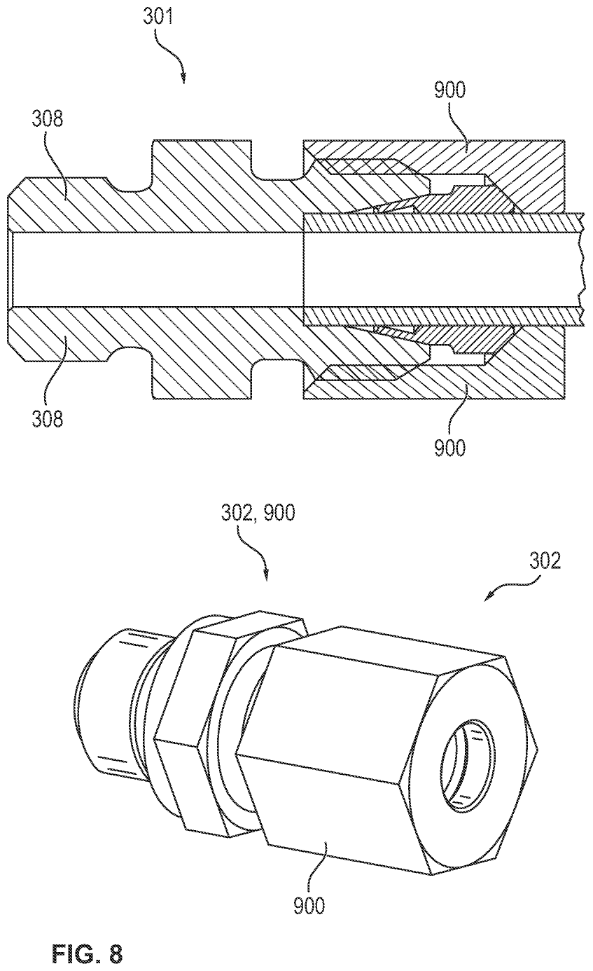

FIGS. 4 and 5 show variants of gas inlet 20 and gas outlet 30 with an outer circular shape (contour) for connecting connecting elements which receive the outer circular shape of gas inlet 20 and gas outlet 30 on the inside. For example, a connection element 900 (FIG. 7, FIG. 8), configured as a cutting ring clamping connection, can receive the outer circular shape of gas outlet 30 on the inside. Gas inlet 20 and gas outlet 30 can be formed with contours on the inside.

Such contours make it possible to avoid additional and possibly complex support structures, as may be required for some manufacturing or joining processes, in particular 3D printing technology processes. For example, 3D printing technologies such as Direct Metal Laser Sintering (DMLS) or Select Laser Melting (SLM) would normally require support structures, i.e. processes in which metal powder is welded in the powder bed by means of a laser.

FIG. 6 shows exemplary configurations of inner contours of gas outlet 30 and gas inlet 20 according to FIG. 1. Identical elements in FIGS. 1, 2, 3, 4, 5 and FIG. 6 are designated with the same reference numerals in FIGS. 1, 2, 3, 4, 5, 6. A detailed illustration 303 shows the gas inlet 20 according to FIG. 1 with internal triangular structures 305, which may preferably be in the form of an equilateral triangle.

A further detailed illustration 304 shows the gas outlet 30 according to FIG. 1 with drop-shaped structures 306 on the inside. The structures 305, 306 according to FIG. 6 make it possible, for example, that in a configuration of the device with an arrangement for pressure reduction in an SLS (selective laser sintering) or SLM (selective laser melting) manufacturing process, no support structures are required inside the gas inlet 20 or gas outlet 30. The layer build-up directions Z 880 for a 3D printing configuration of the gas outlet 30 or the gas inlet 20 are also shown in FIG. 6.

FIG. 7 shows an exemplary embodiment of a gas outlet 30 and/or a gas inlet 20 according to FIG. 1 in an assembly with a connecting element 900. Identical elements in FIGS. 1, 2, 3, 4, 5, 6 and FIG. 7 are designated with the same reference numerals in FIGS. 1, 2, 3, 4, 5, 6, 7. The connecting element 900 is configured as a so-called cutting ring compression fitting. A central axis 390 is shown for illustrative purposes. A transition area 70 is formed within the gas outlet 30, which is configured to provide a smooth transition for the flow in case of a diameter reduction from the mixing system 400, 4000 (FIG. 1, FIG. 3) or the arrangement 300, 800 (FIG. 4, FIG. 5) towards the gas outlet 30. The connecting element 900 receives the gas outlet 30 internally. Cutting clamp elements, as indicated in FIG. 7, provide and ensure a force-locking and form-locking connection of gas outlet 30 and/or gas inlet 20 with the connecting element 900 at one end of the connecting element 900. In this FIG. 7, as well as in FIG. 8, the connection element 900 has, by way of example, at the other end a plug element 308 which is configured for connection to a matching coupling element to enable, for example, a hose line for connection to gas supply sources (FIGS. 1 to 3).

FIG. 8 shows the connecting element 900 in an exemplary embodiment of a cutting ring compression fitting as a plug element 308 according to FIG. 7 in a sectional view 301 as well as in a perspective view 302. Identical elements in FIGS. 1, 2, 3, 4, 5, 6, 7 and FIG. 8 are designated with the same reference numerals in FIGS. 1, 2, 3, 4, 5, 6, 7, 8.

FIG. 9 schematically shows different flow calming elements 600, each as parts of the arrangement 300 according to FIG. 4. Identical elements in FIGS. 1, 2, 3, 4, 5, 6, 7, 8 and FIG. 9 are designated by the same reference numerals in FIGS. 1, 2, 3, 4, 5, 6, 7, 8, 9. Shown are a flow resistance element 601, an orifice element 602, a laminar flow element (LFE) 603 and an impact element 604. These four examples 601, 602, 603, 604 for elements 600 as well as the interfaces 401, 402, 403, 404, 405 (FIG. 4, FIG. 5) can be integrally manufactured gas-tight in a manufacturing or joining process, which is based on a formation of a form-fitting and/or force-fitting as well as gas-tight connection of plastic materials and/or metallic materials based on a printing technology or 3D printing technology.

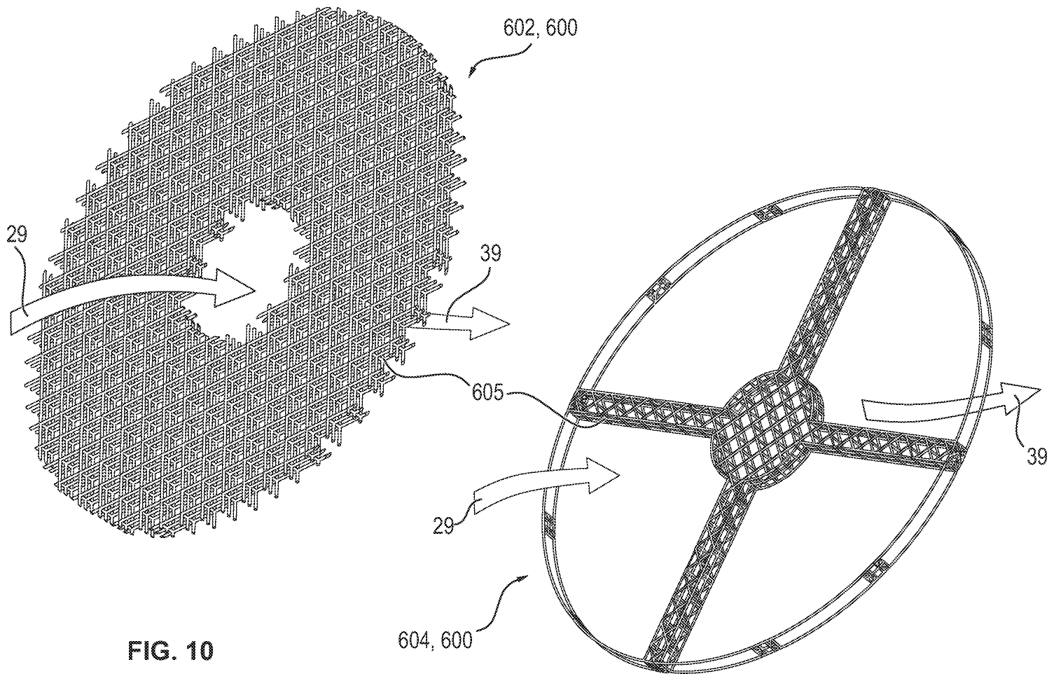

FIG. 10 shows possibilities for an embodiment of elements 600 for flow calming, such as an orifice element 602, as well as a baffle element 604 according to FIG. 9, produced by means of a manufacturing or joining process based on a printing technology or 3D printing technology. Identical elements in FIGS. 1, 2, 3, 4, 5, 6, 7, 8, 9 and FIG. 10 are designated with the same reference numerals in FIGS. 1, 2, 3, 4, 5, 6, 7, 8, 9, 10. In FIG. 10, support structures 605, which may be required to form internally round geometries in manufacturing processes using 3D printing technology, are used to form functional structures, such as elements 600 for flow calming. In 3D printing, the final formation of round shapes usually involves the removal of support structures 605 in a further step. In the embodiment of elements 600 for flow calming, the removal of the support structures in the flow channel 750 (FIGS. 4 and 5) can be partially omitted, so that the support structures 605 themselves can form a suitable shape for flow calming. The sections of the flow channel 750 (FIGS. 4 and 5) in which no elements 600 are to be provided for flow calming can be formed with internal structures 305, 306 (FIG. 6), for example triangular or drop-shaped, using the 3D printing process, as is shown schematically in FIG. 6 in detailed representations 303, 304 as an example for the gas inlets 20, 21 (FIGS. 1-6) and/or the gas outlets 30 (FIGS. 1-6). Such combinations of partial areas with or without support structures clearly illustrate the advantages of 3D printing processes for the configuration of devices for calming or mixing gases.

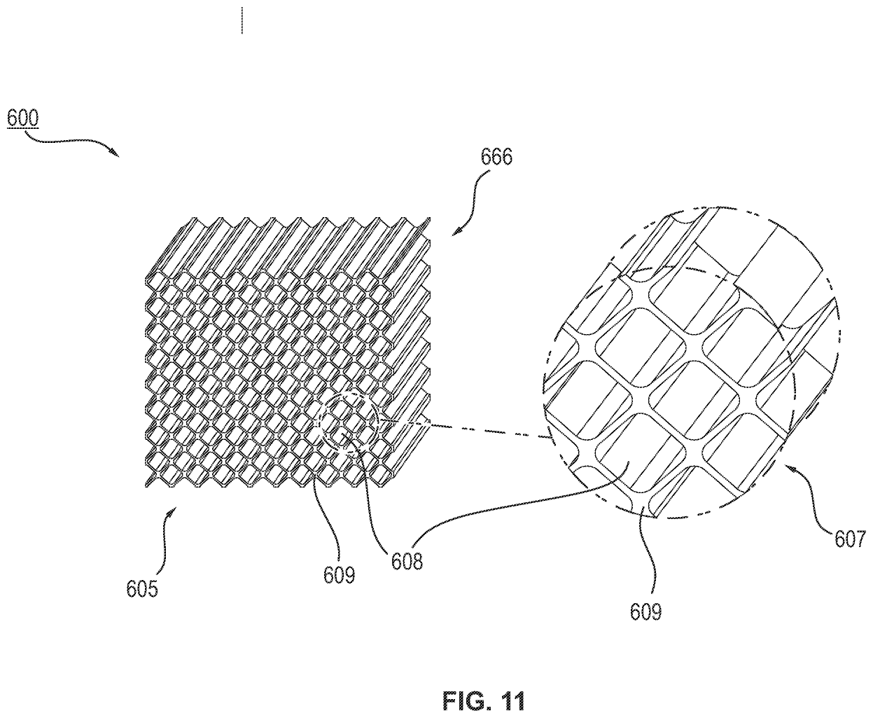

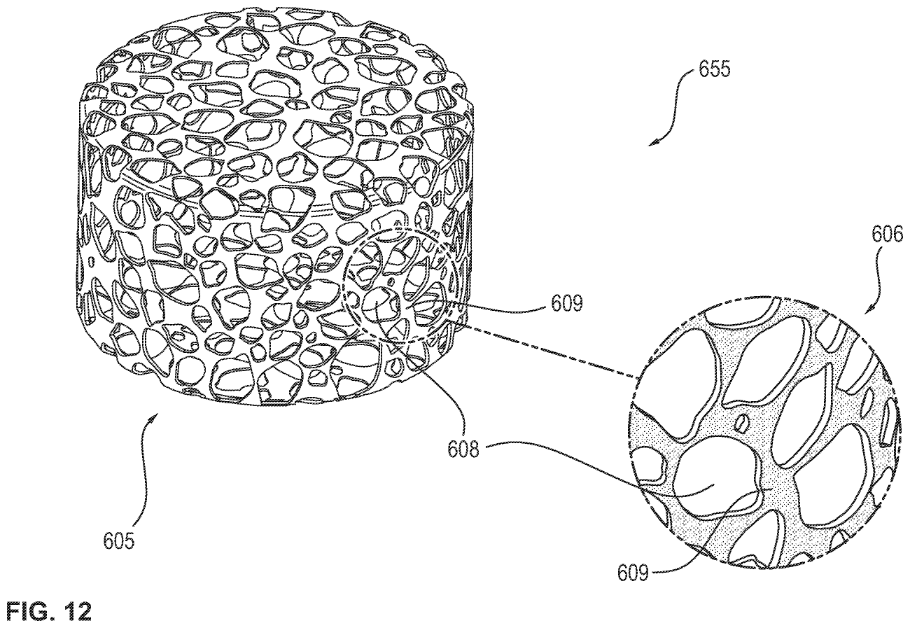

FIG. 11 and FIG. 12 show two examples 655, 666 of embodiments of support structures 605 having cavities 608 and load bearing structures 609. Identical elements in FIGS. 1, 2, 3, 4, 5, 6, 7, 8, 9, 10 and FIGS. 11 and 12 are designated by the same reference numerals in FIGS. 1, 2, 3, 4, 5, 6, 7, 8, 9, 10, 11, 12.

FIG. 11 shows an embodiment of a support structure 605 as a honeycomb support structure 666 with cavities 608 in a drop-shaped truss 608. The truss 609 may also be referred to as a supporting structure 609 or a load-bearing structure 609. Illustration 607 shows, by way of example and in detail, some of the cavities 608 arranged in a regular manner in the supporting structure 609.

FIG. 12 shows an embodiment of a support structure 605 in a free-form 655 having cavities 608 with surrounding load-bearing structures 609. Illustration 606 shows a detail of some cavities 608 and surrounding load-bearing structures 609 in an exemplary irregular arrangement.

While specific embodiments of the invention have been shown and described in detail to illustrate the application of the principles of the invention, it will be understood that the invention may be embodied otherwise without departing from such principles.

### LIST OF REFERENCE NUMBERS

20, 21, 22 Gas inlets (inputs)
25, 26, 28 Gas supply sources
29 Flow arrows from incoming gases
30 Gas outlet (output)
33 Mixed gas inlet at the breathing system
35 Respiratory (breathing) system
37 Anesthesia system
39 Flow arrows of escaping gases
41 First pressure level P1
43 Inlet (input) gas
44 Mixed gas
45 Outlet (output) gas
51 Second pressure level P2
100, 100′, 100″ Devices
200, 2000 Mixing geometry
300, 800 Arrangement
301 Section display
302 Perspective view
303, 304, 606,607 Detailed view
305 Triangular structure
306 Droplet (tear drop) shape structure
308 Plug element
390 Center axis
400, 4000 Mixing system
401-405 Interfaces
501-504 Sensors
505, 506 Actuators
507 Control line
508 Control unit, electronics, μC,
600 Element for flow calming
601 Flow resistance element, flow resistance
602 Orifice element, orifice plate
603 Laminar flow element (LFE)
604 Baffle element, baffle plate
605 Support structures
608 Cavities (hollows)
609 Supporting structure, truss
610 Element to a volume buffering, buffer volume
655 Freeform support structure
666 Honeycomb support structure,
701 Connection to gas state measurement
702 Measuring gas line
703 A gas state measurement unit,
750 Current channel
880 Layer buildup direction Z
900 Connection element, cutting ring-clamping connection

What is claimed is:

1. A device for calming gases or gas mixtures or mixing gases or gas mixtures for an anesthesia system, the device comprising:
a mixing system comprising an integrated mixing geometry;
a plurality of gas inlets, the gas inlets each for supplying a gas or a gas mixture; and
a gas outlet for providing an output of a mixed gas,
wherein the mixing system is fluidically connectable to at least two gas supply sources by means of the gas inlets,
wherein the mixing system is fluidically connectable to at least one mixed gas inlet of a breathing system of the anesthesia system by means of the gas outlet,
wherein the device comprising the gas inlets, the gas outlet and the mixing system, comprising the integrated mixing geometry, is manufactured as a single piece component, integrally and gas-tight in a manufacturing or joining process which is based on a formation of a form-fitting and/or force-fitting as well as gas-tight connection of plastic materials and/or metallic materials based on a printing technology or 3D printing technology.

2. A device according to claim 1, wherein the mixing system comprises an interface for a connection of sensors, for a connection of actuators or for a connection to a gas state measurement device.

3. A device according to claim 2, wherein an element for flow calming, a flow resistance, a flow resistance element, an orifice, an orifice element, a laminar flow element, a baffle element and/or an element for a volume buffering is arranged in the mixing system or in or at the interface.

4. A device according to claim 2, wherein the manufacturing or joining process based on 3D printing technology for manufacturing the interface, the gas inlets and the gas outlet forms the interface, the gas inlets and the gas outlet without use of support structures.

5. A device according to claim 1, wherein an element for flow calming, a flow resistance, a flow resistance element, an orifice, an orifice element, a laminar flow element, a baffle element and/or an element for a volume buffering is arranged in the mixing system.

6. A device according to claim 1, wherein the manufacturing or joining process based on 3D printing technology for manufacturing the gas inlets and the gas outlet forms the gas inlets and the gas outlet without use of support structures.

7. A device according to claim 3, wherein in the manufacturing or joining process based on 3D printing technology, support structures are used for manufacturing the element for flow calming.

8. A device according to claim 2, wherein the interface is configured to measure pressure.

9. A device according to claim 1, wherein:
the gas outlet is configured on an inside with a geometry which forms an inner contour having no step or shoulder in a transition between the gas outlet and the breathing system so that, for a supply of quantities of gas from the gas outlet to the breathing system, a flow state is provided without a flow stall and/or with an essentially locally laminar flow at the transition of the gas outlet to the breathing system; and
the inner contour is produced in the manufacturing or joining process which is based on the formation of a form-fitting and/or force-fitting as well as gas-tight connection of plastic materials and/or metallic materials using the printing technology or 3D printing technology.

10. A device according to claim 1, wherein:
the gas outlet and/or the gas inlets are formed on an outside with a cylindrical or tubular outer contour;
the cylindrical or tubular outer contour is produced in the manufacturing or joining process which is based on the formation of the form-fitting and/or force-fitting and gas-tight connection of plastic materials and/or metallic materials using the printing technology or 3D printing technology.

11. A device according to claim 1, wherein:
the mixing system comprises integral elements that form a common component with one another, the integral elements comprising at least two of functional components of the group comprising: the gas inlets; the gas outlet, an interfaces element, an element to settle flow, a laminar flow element, a flow resistance element, an orifice element, an impact element, and an element to provide a buffer volume;
the integral elements that form a common component with one another are produced in the manufacturing or joining process based on the printing technology or 3D printing technology; and
materials made of plastic materials, plastic composites, metal materials or metal composites are used in the manufacturing or joining process based on the printing technology or the 3D printing technology.

12. A device for a calming of flowing gases or gas mixtures, the device comprising:
a gas inlet for a supply of a gas or a gas mixture;
a flow channel comprising a mixing system comprising an integrated mixing geometry; and
a gas outlet for providing a mixed gas output,
wherein the device comprising the gas inlet, the gas outlet, the flow channel, comprising the mixing system with the mixing geometry, is manufactured as a single piece component, integrally and gas-tight in a manufacturing or joining process which is based on a formation of a form-fitting and/or force-fitting as well as gas-tight connection of plastic materials and/or metallic materials based on a printing technology or 3D printing technology.

13. A device according to claim 12, wherein the mixing system comprises an interface for a connection of sensors, for a connection of actuators or for a connection to a gas state measurement device.

14. A device according to claim 13, wherein an element for flow calming, a flow resistance, a flow resistance element, an orifice, an orifice element, a laminar flow element, a baffle element and/or an element for a volume buffering is arranged in the mixing system or in or at the interface.

15. A device according to claim 13, wherein the manufacturing or joining process based on 3D printing technology for manufacturing the interface, the gas inlets and the gas outlet forms the interface, the gas inlets and the gas outlet without use of support structures.

16. A device according to claim 12, wherein an element for flow calming, a flow resistance, a flow resistance element, an orifice, an orifice element, a laminar flow element, a baffle element and/or an element for a volume buffering is arranged in the mixing system.

17. A device according to claim 12, wherein the manufacturing or joining process based on 3D printing technology for manufacturing the gas inlets and the gas outlet forms the gas inlets and the gas outlet without use of support structures.

18. A device according to claim 12, wherein in the manufacturing or joining process based on 3D printing technology, support structures are used for manufacturing the flow calming element.

19. A device according to claim 13, wherein the interface is configured to measure pressure.

20. A device according to claim 12, wherein:
the gas outlet is configured on an inside with a geometry which forms an inner contour having no step or shoulder in a transition between the gas outlet and the breathing system so that, for a supply of quantities of gas from the gas outlet to the breathing system, a flow state is provided without a flow stall and/or with an essentially locally laminar flow at the transition of the gas outlet to the breathing system; and
the inner contour is produced in a manufacturing or joining process which is based on the formation of a form-fitting and/or force-fitting as well as gas-tight connection of plastic materials and/or metallic materials using the printing technology or 3D printing technology.

21. A device according to claim 12, wherein:
the gas outlet and/or the gas inlets are formed on an outside with a cylindrical or tubular outer contour;
the cylindrical or tubular outer contour can be produced in the manufacturing or joining process which is based on the formation of the form-fitting and/or force-fitting and gas-tight connection of plastic materials and/or metallic materials using the printing technology or 3D printing technology.

22. A device according to claim 12, wherein:
the mixing system comprises integral elements that form a common component with one another, the integral elements comprising at least two of functional components of the group comprising: the gas inlets; the gas outlet, an interfaces element, an element to settle flow, a laminar flow element, a flow resistance element, an orifice element, an impact element, and a element to provide a buffer volume;
the integral elements that form a common component with one another are produced in a manufacturing or joining process based on the printing technology or 3D printing technology; and
materials made of plastic materials, plastic composites, metal materials or metal composites are used in the manufacturing or joining process based on the printing technology or the 3D printing technology.

23. A process of automated additive manufacturing of a device, the process comprising the steps of:
forming the device so as to comprise, as a single common component, at least two of the following functional components: a gas inlet; another gas inlet; a gas outlet; an interface; a flow calming element to settle the flow; a laminar flow element; a flow resistance element; an orifice element; an impact element; and a gas volume buffering element,
wherein the common component is formed in a manufacturing or joining process based on a printing technology or 3D printing technology, and
wherein materials made of plastic materials, plastic composites, metal materials or metal composites are used in the manufacturing or joining process.

24. A process according to claim 23, wherein the manufacturing or joining process based on 3D printing technology manufactures one or more of the interface, gas inlets and the gas outlet without the use of support structures.

25. A process according to claim 23, wherein in the manufacturing or joining process based on 3D printing technology, one or more support structures are used for manufacturing one or more flow calming elements.

26. A process according to claim 23, wherein a computer program or computer program product, comprising program code provided with a non-transitory computer-readable media, is executable to perform or control a production device for the manufacturing or joining process based on a printing technology or 3D printing technology.
